(19) United States
(12) Patent Application Publication
(10) Pub. No.: US 2020/0030164 A1
(43) Pub. Date: Jan. 30, 2020

(54) Securing Member

(71) Applicant: 3M INNOVATIVE PROPERTIES COMPANY, St. Paul, MN (US)

(72) Inventors: Yoshihisa Matsuda, Sagamihara (JP); Hirofumi Hosokawa, Tokyo (JP); Kioshi Kunihiro, Sagamihara (JP); Makoto Nagase, Tokyo (JP); Hiroyasu Nagata, Tokyo (JP); Masashi Sonoda, Tokyo (JP)

(21) Appl. No.: 16/478,219

(22) PCT Filed: Jan. 26, 2018
(86) PCT No.: PCT/IB18/50499

§ 371 Date: Jul. 16, 2019

(30) Foreign Application Priority Data
Feb. 1, 2017 (JP) 2017-017017

(51) Int. Cl. A61F 13/551; A61F 13/58

(52) U.S. Cl. CPC A61F 13/5512; A61F 13/581

(57) ABSTRACT

A securing member is provided with a first, second, and third strip member, and the strip members are folded and laminated. The first strip member has a strip-shaped first substrate layer and a first adhesive layer provided on a first surface of the first substrate layer; the second strip member has a strip-shaped second substrate layer positioned between the first and third strip members, and a third adhesive layer provided on a first surface of the second substrate layer; and the third strip member has a strip-shaped third substrate layer and a third adhesive layer provided on a first surface of the third substrate layer. A portion of the first adhesive layer is detachably adhered to a second surface of the second substrate layer; a portion of the second adhesive layer is detachably adhered to a second surface of the third substrate layer; and the second substrate layer has elasticity.

### TECHNICAL FIELD

One aspect of the present invention relates to a securing member.

### BACKGROUND ART

A securing member used for assembling a target article is conventionally known. For example, the following Patent Document 1 discloses a composite adhesive tape formed from three tapes including a first tape, second tape, and third tape. In the composite adhesive tape, the first tape and second tape, and the second tape and third tape are respectively combined by adhering at an end portion along a longitudinal direction. Furthermore, a cross section orthogonal to the longitudinal direction of the composite adhesive tape is folded into a Z shape such that the first tape corresponds to a Z-shaped horizontal first stroke, the second tape corresponds to a diagonal second stroke, and the third tape corresponds to a horizontal third stroke.

In a case where the composite adhesive tape is used, first, the third tape is secured to a target article, and then the folded condition of the first to third tapes is undone. Next, the target article is assembled by winding the first to third tapes on the target article. Next, an adhesive provided on the first to third tapes is adhered onto the target article to maintain a condition where the target article is assembled.

### CITATION LIST

### Patent Literature

Patent Document 1: JP 2636103 B

### SUMMARY OF THE INVENTION

In a case where the target article is assembled using a securing member such as the aforementioned composite adhesive tape, the adhesive of the second tape that is not structurally adhered on the target article may adhere to a worker or another article. In this case, the assembled target article needs to be handled such that the adhesive is not adhered to another article or the like, and thus processing of the target article becomes complicated. Therefore, in the securing member assembling the target article, an aspect is desired where occurrences of adhering to an article or the like other than the target article can be suppressed.

### Means for Solving the Problem

A securing member according to one aspect of the present invention, includes: a first strip member; a second strip member; a third strip member; a first bonding portion of the second strip member and first strip member; and a second bonding portion of the second strip member and third strip member; the first to third strip members being folded and laminated; wherein the first strip member has a strip-shaped first substrate layer and a first adhesive layer provided on a first surface of the first substrate layer; the second strip member has a strip-shaped second substrate layer positioned between the first strip member and the third strip member, and a second adhesive layer provided on a first surface of the second substrate layer; the third strip member has a strip-shaped third substrate layer and a third adhesive layer provided on a first surface of the third substrate layer; at least a portion of the first adhesive layer is detachably adhered to a second surface of the second substrate layer, at least a portion of the second adhesive layer is detachably adhered to a second surface of the third substrate layer, and the second substrate layer has elasticity, and a width of at least a portion thereof narrows when a tensile load is applied in one direction.

According to this aspect, the third adhesive layer is adhered to the member to be secured, and then the first adhesive layer is detached from the second substrate layer, and the second adhesive layer is detached from the third substrate layer. Thereby, a crease on the first bonding portion and second bonding portion is eliminated, the first to third strip members form an integral strip shape, and the securing member can be wound on the member to be secured with the third strip member as a starting point. Furthermore, the first adhesive layer is adhered to the member to be secured in a condition where the securing member is wound on the member to be secured, and therefore, the member to be secured can be favorably assembled by the securing member. At this time, the second substrate layer is elastically deformed by applying a tensile load, and therefore, the securing member is wound on the member to be secured in a condition where a width of at least a portion thereof is narrow. Thereby, the second adhesive layer is compressed, at least a portion thereof is wrapped in the second substrate layer, and an adhesive force of the second adhesive layer is reduced. Therefore, the first to third strip members are formed into an integral strip shape, and thus even in a case where the second adhesive layer is not structurally adhered to the member to be secured, the securing member can be suppressed from adhering to an article other than the member to be secured.

A securing member according to another aspect of the present invention, includes: a first strip member; a second strip member; a third strip member; a first bonding portion of the second strip member and first strip member; and a second bonding portion of the second strip member and third strip member; the first to third strip members being folded and laminated; wherein the first strip member has a strip-shaped first substrate layer and a first adhesive layer provided on a first surface of the first substrate layer; the second strip member has a strip-shaped second substrate layer positioned between the first strip member and the third strip member; the third strip member has a strip-shaped third substrate layer and an adhesive layer provided on a first surface of the third substrate layer; and at least a portion of the first adhesive layer is detachably adhered to the second substrate layer and a second surface of the third substrate layer.

According to this aspect, at least a portion of the first adhesive layer is detachably adhered to the second substrate layer and the second surface of the third substrate layer. Therefore, even in a case where an adhesive layer is not provided on the second strip member, a laminated condition of the first to third strip members before use is maintained by the first adhesive layer. Furthermore, the adhesive layer is adhered to the member to be secured, and then the first adhesive layer is detached from the second substrate layer and third substrate layer. Thereby, a crease on the first bonding portion and second bonding portion is eliminated, the first to third strip members form an integral strip shape, and the securing member can be wound on the member to be secured with the third strip member as a starting point. Furthermore, the first adhesive layer is adhered to the member to be secured in a condition where the securing member is wound on the member to be secured, and therefore, the member to be secured can be favorably assembled by the securing member. Therefore, according to the aforementioned aspect, the first to third strip members can be formed into an integral strip shape such that the second strip member has adhesion, and thus the securing member can be suppressed from adhering to an article other than the member to be secured.

### Effect of the Invention

One aspect of the present invention can suppress the occurrences of adhering to an article or the like other than a member to be secured.

### BRIEF DESCRIPTION OF THE DRAWINGS

FIG. 1(a) is a plan view of a securing member according to a first embodiment, and FIG. 1(b) is a view illustrating a cross section along line Ib-Ib in FIG. 1(a).

FIG. 2(a) is an expanded view of the securing member according to the first embodiment, and FIG. 2(b) is a view illustrating a cross section along line IIb-IIb in FIG. 2(a).

FIG. 3(a) is a schematic plan view illustrating a latching portion, and FIG. 3(b) is a view illustrating a cross section along line in FIG. 3(a).

FIG. 4 is a view illustrating a condition when an opened securing member is stretched.

FIG. 5 is a view illustrating a sheet as a base for the securing member according to the first embodiment.

FIG. 6 is a schematic view illustrating a condition where the securing member in a laminated condition is adhered to a member to be secured.

FIG. 7 is a view illustrating a condition where the securing member in an opened condition is wound and secured on the member to be secured.

FIG. 8(a) is a view illustrating an opened condition of the securing member according to a first modified example of the first embodiment, and FIG. 8(b) is a view illustrating an opened condition of the securing member according to a second modified example of the first embodiment.

FIG. 9(a) is a plan view of a securing member according to a second embodiment, and FIG. 9(b) is a view illustrating a cross section along line IXb-IXb in FIG. 9(a).

FIG. 10(a) is a view illustrating a cross section of a securing member according to a third embodiment, and FIG. 10(b) is a view illustrating a cross section of a securing member according to a fourth embodiment.

FIG. 11 is a view illustrating a cross of a securing member according to a fifth embodiment.

FIG. 12 is a diagram showing a hysteresis loop when the second substrate layer of Examples 1 to 3 is stretched and contracted.

### DESCRIPTION OF THE PREFERRED EMBODIMENTS

Preferred embodiments of the present invention will be described below while referring to the drawings. Note that the same elements are denoted with the same reference numerals in the description of the drawings, and duplicate descriptions are omitted. Furthermore, in order to facilitate understanding, the drawings are exaggeratedly drawn, and the dimensional ratios and the like are not limited to those described in the drawings.

“Securing member” in the present specification refers to a member used to secure a member to be secured. The member to be secured may be an arbitrary article, the type, shape, dimensions, number, and the like are in no way limited. Therefore, the purpose of use of the securing member is also not limited.

As one example, the securing member is used to retain the shape of the member to be secured that is assembled to be compact by rounding, folding, or winding. For example, the securing member can be used to retain a condition of an absorbent article (for example, hygienic products such as a disposable diaper, catamenial napkin, incontinence pad, and the like) assembled to be compact. As another example, the securing member may be used to mutually secure the same type or different types of members to be secured. For example, the securing member may be used to attach or secure an absorbent article to clothing, or may be used to bundle a plurality of articles. In other words, the member to be secured is not limited to a single article. Furthermore, as another example, the securing member may be used to secure a first region and a second region on a member to be secured. For example, the securing member may be used as a sealing tape attached to a food product bag and used for storing food products.

The securing member is provided with a member (strip member) having a plurality of strip shapes. The term “strip member” in the present specification refers to a long and thin plate-shaped, rod-shaped, or string-shaped member. In the present specification, a direction orthogonal to a longitudinal direction A of the strip member is defined as a width direction B. The longitudinal direction A can also be referred to as a winding direction of the securing member, or opening direction (one direction) of the securing member. The strip members linearly extend along the longitudinal direction A. In a certain aspect, a length (long side length) of the strip members in the longitudinal direction A may be from 5 to 100 m, from 10 to 75 mm, or from 15 to 50 mm, a length (short side length) of the strip members in the width direction B may be from 2 to 30 mm, from 3 to 25 mm, or from 4 to 20 mm, and a thickness of the strip members may be from 30 to 300 μm, from 40 to 250 μm, or from 50 to 200 μm.

### First Embodiment

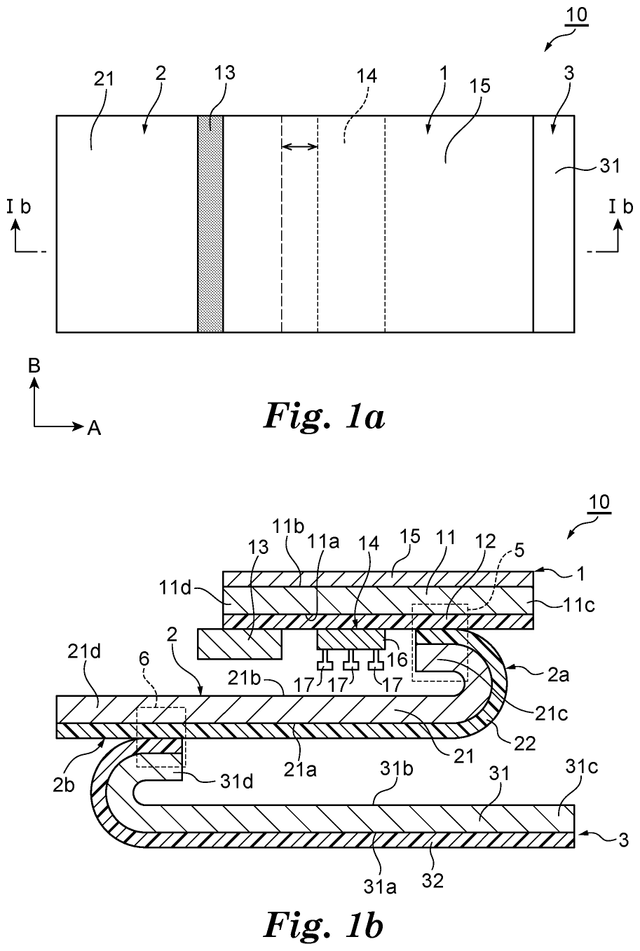

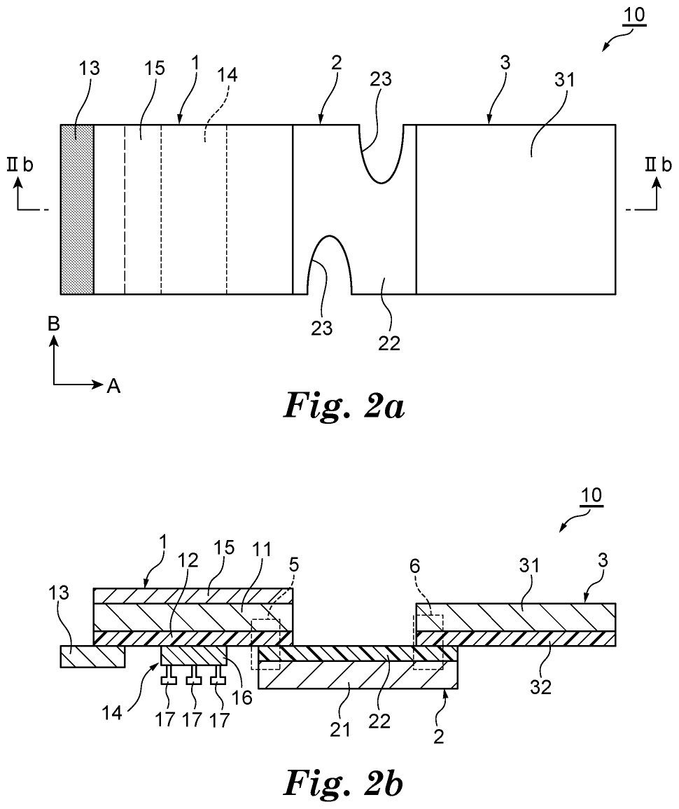

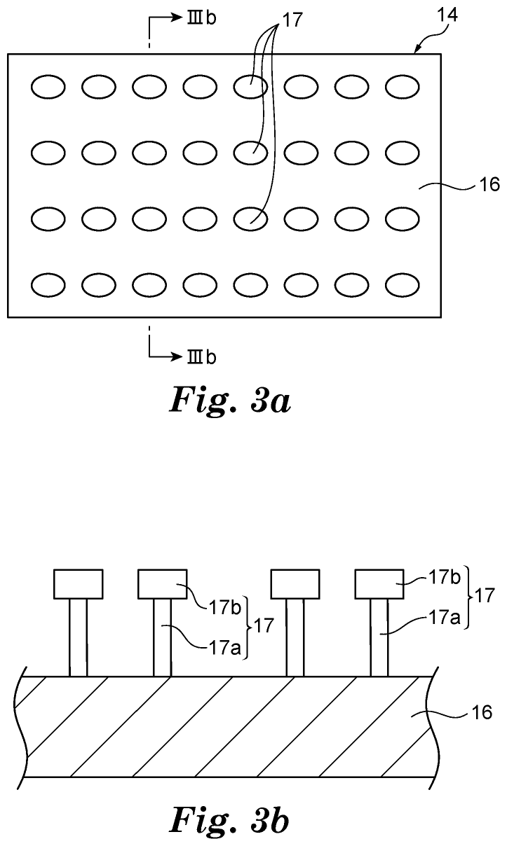

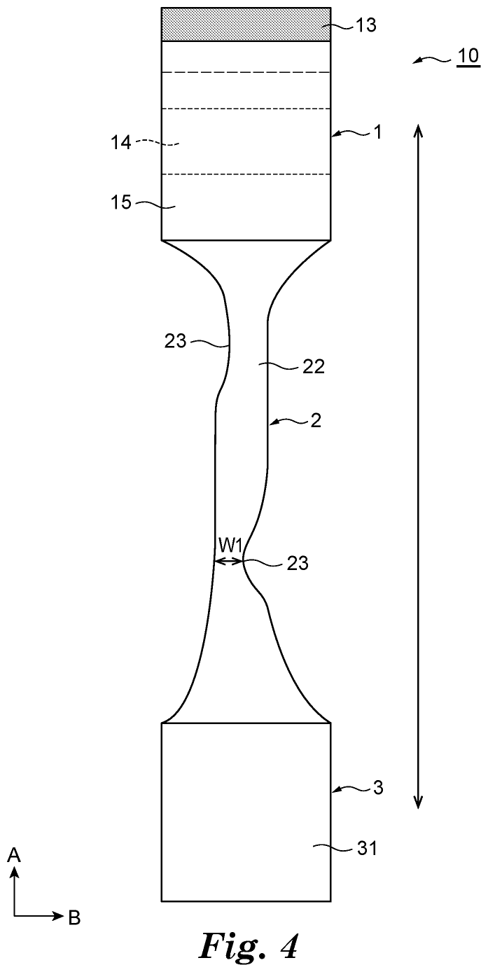

First, a configuration and structure of a securing member according to a first embodiment will be described while referring to FIGS. 1(a) to 4. FIG. 1(a) is a plan view of the securing member according to the first embodiment, and FIG. 1(b) is a view illustrating a cross section along line Ib-Ib in FIG. 1(a). FIG. 2(a) is an expanded view of the securing member according to the first embodiment, and FIG. 2(b) is a view illustrating a cross section along line IIb-IIb in FIG. 2(a). FIG. 3(a) is a schematic plan view illustrating a latching portion, and FIG. 3(b) is a view illustrating a cross section along line in FIG. 3(a). FIG. 4 is a view illustrating a condition when an opened securing member is stretched.

A securing member 10 according to the first embodiment has an essentially Z-shaped cross section in conjunction with having a strip shape in plan view, as illustrated in FIGS. 1(a) and 1(b). The securing member 10 is provided with a mutually laminated first strip member 1, second strip member 2, and third strip member 3. Specifically, the securing member 10 is provided with a first strip member 1 corresponding to an upper side of a Z shape, a second strip member 2 corresponding to an inclined line of the Z shape, and a third strip member corresponding to a lower side of the Z shape, in a cross section. Therefore, in the securing member 10, the first strip member 1, second strip member 2, and third strip member 3 are laminated in order. The securing member 10 has a first bonding portion 5 of the first strip member 1 and a first end portion 2a of the second strip member 2, and a second bonding portion 6 between the third strip member 3 and a second end portion 2b of the second strip member 2 as creases, and the first strip member 1, second strip member 2, and third strip member 3 are folded and laminated. Therefore, in the longitudinal direction A, the first bonding portion 5 is positioned on a first end side of the securing member 10, and the second bonding portion 6 is positioned on a second end side of the securing member 10.

Hereinafter, a condition where the first strip member 1, second strip member 2, and third strip member 3 of the securing member 10 are folded and laminated is defined as a “laminated condition”, and a direction where the first strip member 1, second strip member 2, and third strip member 3 are laminated is defined as a “laminating direction”. Note that in FIG. 1(b), for the purpose of description, a large portion of the first strip member 1 and second strip member 2 and a large portion of the second strip member 2 and third strip member 3 are not in close contact in the laminating direction, but these portions are actually in close contact with each other in the laminating direction. A long side length of the securing member 10 in the laminated condition is 5 mm or greater, 10 mm or greater, or 15 mm or greater, and 50 mm or less, 45 mm or less, or 40 mm or less, for example.

In the securing member 10, the aforementioned creases can be undone, and the first strip member 1, second strip member 2, and third strip member 3 can be opened. For example, the first strip member 1 is pulled in the laminating direction while securing the third strip member 3. Furthermore, the first strip member 1, second strip member 2, and third strip member 3 can be opened by undoing the creases in conjunction with eliminating close contact between the first strip member 1 and second strip member 2 other than the first bonding portion 5, and close contact between the second strip member 2 and third strip member 3 other than the second bonding portion 6. Thereby, as illustrated in FIGS. 2(a) and 2(b), the opened securing member 10 has an integral strip shape with the first strip member 1, second strip member 2, and third strip member 3 arranged in order. Hereinafter, a condition where the securing member 10 has an integral strip shape is defined as an “opened condition”. The securing member 10 is wound on the member to be secured in the opened condition to retain the shape of the assembled member to be secured. Note that in the following description, a securing member 10 in a laminated condition is described for each component of the securing member 10 unless specified as a “case where the securing member 10 is in the opened condition” or the like. Note that FIG. 2(b) illustrates a condition where the securing member 10 is opened when the third strip member 3 is adhered to the member to be secured. When the third strip member 3 is adhered to the member to be secured, in contrast to FIG. 2(b), the second strip member 2 does not wrap around to a lower side than the third strip member 3, and the second bonding portion 6 also does not reach horizontal (in the drawing).

The first strip member 1 is a portion configuring an frontmost surface in a case where the securing member 10 is secured to the member to be secured in a laminated condition. Furthermore, the first strip member 1 is a portion which is a first end in the longitudinal direction A, secured to the member to be secured for the opened securing member 10. The first strip member 1 has a substrate layer 11 (first substrate layer), adhesive layer 12 (first adhesive layer), tab portion 13, latching portion 14, and outermost layer 15.

The substrate layer 11 is a member having a strip shape, and has a surface 11 (first surface) facing a second strip member 2 side (and third strip member 3 side), and a surface 11b (second surface) on a side opposite from the surface 11a. The adhesive layer 12, tab portion 13, and latching portion 14 are provided on the surface 11a. The outermost layer 15 is provided on the surface 11b. As viewed from the laminating direction, a long side length of the substrate layer 11 is from 5 mm to 100 mm, and a short side length of the substrate layer 11 is from 2 mm to 30 mm. A thickness of the substrate layer 11 is from 20 μm to 200 μm, for example.

A first end portion 11c of the substrate layer 11 in the longitudinal direction A is a portion configuring the aforementioned first bonding portion 5. Furthermore, one portion of the end portion 11c of the substrate layer 11 is overlaid with a first end portion 2a of the second strip member 2 in the laminating direction. In other words, a tip end of the end portion 11c in the longitudinal direction A is positioned more on the outside than the first end portion 2a of the second strip member 2. A second end portion 11d of the substrate layer 11 in the longitudinal direction A is positioned between the first bonding portion 5 and second bonding portion 6 in the longitudinal direction A. The end portion 11d is provided so as to not overlay with the first bonding portion 5 and second bonding portion 6 in the laminating direction.

The substrate layer 11 is configured from a resin film, paper, or nonwoven material, for example. In a case where the substrate layer 11 is a resin film, the resin configuring the substrate layer 11 is a polyethylene, polypropylene, or other polyolefin, polyester, polyamide, polyurethane, polyvinyl chloride, or other synthetic resin.

The adhesive layer 12 is a layer maintaining the laminated condition of the securing member 10. The adhesive layer 12 is detachably adhered to a substrate layer 21 (described later in detail) of the second strip member 2, and adhered to an adhesive layer 22 (described later in detail) of the second strip member 2 by the first bonding portion 5. Therefore, in a case where the securing member 10 is in a laminated condition, the first strip member 1 and second strip member 2 are in close contact, and a laminated condition thereof is maintained. Furthermore, in the opened securing member 10, the adhesive layer 12 is a layer that adheres the substrate layer 11 and member to be secured. A thickness of the adhesive layer 12 is from 5 μm to 100 μm for example.

The first end portion 2a of the second strip member 2 is adhered to a portion provided on the end portion 11c of the substrate layer 11 for the adhesive layer 12. The tab portion 13 is adhered to a portion provided on the end portion 11d of the substrate layer for the adhesive layer 12. For the adhesive layer 12, the latching portion 14 is adhered between the portion adhered to the first end portion 2a of the second strip member 2 and portion adhered to the tab portion 13.

The adhesive layer 12 is formed by coating a pressure-sensitive adhesive or the like on the surface 11a of the substrate layer 11. The pressure-sensitive adhesive included in the adhesive layer 12 is an acrylic pressure-sensitive adhesive, rubber pressure-sensitive adhesive, urethane pressure-sensitive adhesive, or silicone pressure-sensitive adhesive, for example.

The adhesive force of the first strip member 1 and second strip member 2 (in other words, force applied when the adhesive layer 12 is peeled from the substrate layer 21) is 0.1 N/25 mm or greater or 0.3 N/25 mm, and 10 N/25 mm or less or 5 N/25 mm. When the adhesive force is 0.1 N/25 mm or greater, the first strip member 1 can be prevented from inadvertently opening from the second strip member 2. When the adhesive force is 10 N/25 mm or less, the first strip member 1 can be detached from the second strip member 2 by an appropriate force.

The aforementioned adhesive force corresponds to 90 degree peeling force measured as follows (90 degree peeling force of the first strip member 1 with regard to the second strip member 2), for example, and is an indicator for the ease of opening the first strip member 1 and second strip member 2 in the securing member 10. First, the first strip member 1 is processed to prepare a 25 mm×50 mm test piece. Next, a 25 mm×210 mm paper strip is adhered to half of the test piece (25×25 mm) to provide a sample having a linear shape. Next, the second strip member 2 (substrate layer 21) which is an adherend is adhered to a 50 mm×100 mm aluminum plate. Herein, a long side of the aluminum plate is disposed so as to extend along a sample peeling direction (peeling direction). Next, a portion (25 mm×25 mm) of the sample where the test piece is exposed is adhered to the adherend using a 2 kg roller. Furthermore, the aforementioned adhesive force is measured by peeling the sample from the adherend under 300 mm/min rate condition, using a tensile tester or a device having a similar ability thereto. Note that the 90 degree peeling force is an average value from the start of peeling to a moving distance of 25 mm.

In the opened securing member 10, the adhesive layer 12 adheres the member to be secured and substrate layer 11 by an adhesive force sufficient for retaining the shape of the assembled member to be secured. In a case where a material of the member to be secured is a nonwoven material, the 90 degree peeling force of the adhesive layer 12 with regard to the member to be secured is 0.5 N/25 mm or greater, for example, and a dynamic shear force (also referred to as dynamic shear strength) of the adhesive layer 12 with regard to the member to be secured is 4 N/(25 mm×25 mm) or greater, for example. Note that the 90 degree peeling force of the adhesive layer 12 with regard to the nonwoven material can be similarly measured as the aforementioned 90 degree peeling force of the first strip member 1 with regard to the second strip member 2, except when the adherend adhered to an aluminum plate is a nonwoven material (PS-108-S available from Mitsui Chemicals, Inc.). Furthermore, the dynamic shear force of the adhesive layer 12 with regard to the nonwoven material can be measured as follows, for example. First, the first strip member 1 is processed to prepare a 25 mm×50 mm test piece. Next, a 25 mm×50 mm PET strip (thickness: 100 μm) is adhered to half of the test piece (25×25 mm) to provide a sample having a linear shape. Next, a nonwoven material (PS-108-S available from Mitsui Chemicals, Inc.) which is an adherend is adhered to a 50 mm×100 mm aluminum plate. Herein, a long side of the aluminum plate is disposed so as to extend along a sample peeling direction (peeling direction). Next, a portion (25 mm×25 mm) of the sample where the test piece is exposed is adhered to the adherend using a 2 kg roller. Furthermore, the aforementioned dynamic shear force is measured by pulling the sample in a long side direction (180 degree direction) of the aluminum plate from the adherend under a 30 mm/min rate condition, using a tensile tester or a device having a similar ability thereto. Note that the dynamic shear force is a maximum value of a tensile load.

The tab portion 13 is a portion which serves as a grip portion when the securing member 10 is opened, and is adhered to the adhesive layer 12 so as to protrude from the end portion 11d of the substrate layer 11 in the longitudinal direction A. In other words, the tab portion 13 is provided positioned on the end portion 11d on a side opposite from the end portion 11c configuring the first bonding portion 5 on the first strip member 1. From the perspective of easily determining the position of the tab portion 13 on the securing member 10, the tab portion 13 may be colored. In the longitudinal direction A, the tab portion 13 is exposed from the substrate layer 11 by from approximately 0.1 mm to 20 mm, for example. The tab portion 13 is provided between the first bonding portion 5 and second bonding portion 6 in the longitudinal direction A, similar to the end portion 11d of the substrate layer 11. The tab portion 13 is provided so as to not overlay with the first bonding portion 5 and second bonding portion 6 in the laminating direction. The tab portion 13 is configured from a resin film, paper, nonwoven material, or the like, for example. A thickness of the tab portion 13 is approximately 80 μm for example.

The latching portion 14 is a hook material configured to be physically latchable to the member to be secured when the securing member 10 is opened. For example, in a case where the member to be secured is a nonwoven material (PS-108-S available from Mitsui Chemicals, Inc.), the 90 degree peeling force of the latching portion 14 with regard to the member to be secured is 0.1 N/25 mm or greater, for example, and the dynamic shear force (also referred to as dynamic shear strength) of the latching portion 14 with regard to the member to be secured is 4 N/(25 mm×25 mm) or greater, for example. The 90 degree peeling force and dynamic shear force of the latching portion 14 with regard to the nonwoven material can be similarly measured as the 90 degree peeling force and dynamic shear force of the adhesive layer 12 with regard to the nonwoven material.

As illustrated in FIG. 1(b) and FIGS. 3(a) and 3(b), the latching portion 14 has a structure where a plurality of hooks 17 are provided on a substrate 16. The substrate 16 is a portion having a strip shape and is adhered to the adhesive layer 12. From the perspective of enhancing a latching force to the member to be secured, a length of the substrate 16 along the longitudinal direction A is 2 mm or greater, 3 mm or greater, or 4 mm or greater, for example. From the perspective of maintaining an adhesive force in the laminated condition, a length of the substrate 16 along the longitudinal direction A is 30 mm or less, 20 mm or less, or 10 mm or less, for example. A length of the substrate 16 along the width direction B is approximately the same as the short side length of the substrate layer 11. The hook 17 has an essentially mushroom shape in a side surface view, and has a stem portion 17a extending toward a second strip member 2 side from the substrate 16, and a canopy portion 17b formed on a tip end of the stem portion 17a. The canopy portion 17b has an elliptical shape having a long diameter along the longitudinal direction A as viewed from the laminating direction, for example. Thereby, when a force is generated where the securing member 10 tends to contract in a condition where the latching portion 14 is latched on the member to be secured, the hook 17 easily bites into the member to be secured. In other words, a latching force of the latching portion 14 is more easily demonstrated. Note that the shape of the hook 17 is not limited thereto, and may be appropriately set based on the dimensions (length and width), degree of adjustment, or the like of the securing member. For example, so long as the latching force is satisfied, the hook 17 may have a hook shape, T shape, J shape, or the like.

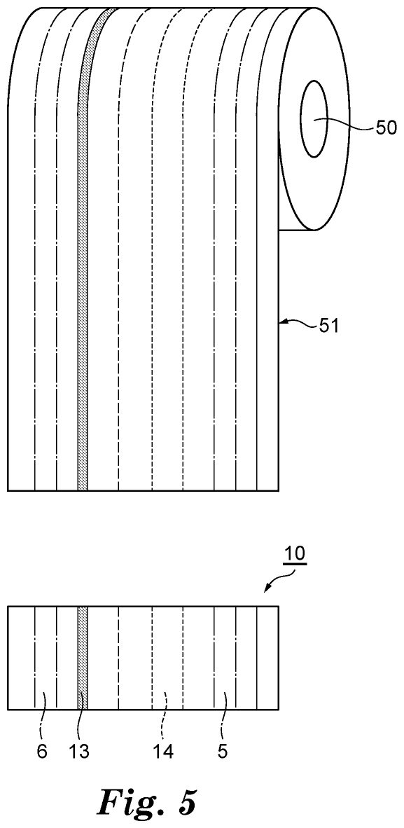

The latching portion 14 is provided between the first bonding portion 5 and the tab portion 13 in the longitudinal direction A. The latching portion 14, the first bonding portion 5, second bonding portion 6, and tab portion 13 do not overlap each other in the laminating direction. Furthermore, in a case where the securing member 10 is in the laminated condition, the latching portion 14 can be prevented from being covered by the first strip member 1 and second strip member 2, and latching to the member to be secured and the like. For example, when viewed from the laminating direction, the ratio of the latching portion 14 with regard to an area of the adhesive layer 12 is 70% or lower or 60% or lower. In this case, the adhesive layer 12 can be suppressed from easily peeling from the second strip member 2 (in other words, the first strip member 1 from being stripped from the second strip member 2) in the laminated condition. Furthermore, from the perspective of maintaining adhesive force in the laminated condition, the latching portion 14 is separated 3 mm or greater, 4 mm or greater, or 5 mm or greater in the longitudinal direction A with regard to an inner side of the tab portion 13 (first bonding portion 5 side), for example. Furthermore, from the perspective of preventing the first strip member 1 from peeling from the tab portion 13 side when latching to the member to be secured, the latching portion 14 is separated 20 mm or less, 15 mm or less, or 10 mm or less in the longitudinal direction A with regard to an inner side of the tab portion 13, for example. In this case, adhesion of the adhesive layer 12 positioned between the tab portion 13 and latching portion 14 is sufficiently demonstrated, and the end portion 11d of the adhesive layer 12 can be suppressed from easily peeling from the second strip member 2. Furthermore, as described later, there is an effect where a specific location is less likely to be thick when wound into a roll shape (refer to FIG. 5 described later).

The securing member 10 can be latched on the member to be secured by the adhesive layer 12 and latching portion 14 by a 90 degree peeling force of at least 0.5 N/25 mm or greater. Furthermore, the securing member 10 can be latched on the member to be secured by the adhesive layer 12 and latching portion 14 by a dynamic shear force of at least 4 N/(25 mm×25 mm) or greater. The 90 degree peeling force and dynamic shear force of the adhesive layer 12 and latching portion 14 with regard to the nonwoven material can be similarly measured as the 90 degree peeling force and dynamic shear force of the adhesive layer 12 with regard to the nonwoven material.

The outermost layer 15 is a portion serving as a surface of the securing member 10 when the securing member 10 is adhered to the member to be secured. The outermost layer 15 is provided so as to cover all of the surface 11b of the substrate layer 11. The outermost layer 15 is secured to the substrate layer 11 through an adhesive or the like not illustrated in the drawing, for example. The outermost layer 15 may be the same material as the substrate layer 11. The outermost layer 15 may be processed so as to approach the texture of the member to be secured. For example, the outermost layer 15 is configured from a resin film where an uneven surface is provided by an embossing process or the like. In a case where the member to be secured is configured from paper or a nonwoven material, the surface thereof is an uneven surface. Therefore, an uneven surface is provided on the surface of the outermost layer 15 positioned above the surface 11b on the substrate layer 11, and therefore, an integral appearance and feel of the securing member 10 and member to be secured are easier to achieve. However, when the nonwoven material or paper is selected as the same material as the securing member for the outermost layer 15, the nonwoven material or paper directly contacts an adhesive layer 32 when wound on a roller as in FIG. 5, and thus problems may occur when unwinding from the roller. In order to prevent this, attaching a peeling layer (liner) to the adhesive layer 32 may be considered, but is disadvantageous from the perspective of cost, man-hours, and the like. Therefore, the outermost layer 15 is preferably configured from a resin film or the like where an uneven surface is provided.

The second strip member 2 is a portion that connects the first strip member 1 and third strip member 3 on the securing member 10, and is interposed between the first strip member 1 and third strip member 3. The second strip member 2 has the substrate layer 21 (second substrate layer) and adhesive layer 22 (second adhesive layer).

The substrate layer 21 is a transparent portion having a strip shape, and has a surface 21a (first surface) facing a third strip member 3 side, and a surface 21b (second surface) facing a first strip member 1 side, on a side opposite from the surface 21a. The adhesive layer 22 is provided above the surface 21a, and the first strip member 1 is positioned above the surface 21b. At least a portion of the adhesive layer 12 of the first strip member 1 is detachably adhered to the surface 21b. As viewed from the laminating direction, a long side length of the substrate layer 21 is 5 mm to 100 mm, and a short side length of the substrate layer 21 essentially the same as the substrate layer 11. A thickness of the substrate layer 21 is from 20 μm to 200 μm for example.

A first end portion 21c of the substrate layer 21 in the longitudinal direction A configures the first end portion 2a of the second strip member 2, and covers the first strip member 1. The end portion 21c is folded toward the first strip member 1 side when the securing member 10 is in the laminated condition. Therefore, a portion of the surface 21a faces the first strip member 1 side on the end portion 21c. A second end portion 21d of the substrate layer 21 in the longitudinal direction A configures the second end portion 2b of the second strip member 2, and is exposed from the first strip member 1 as viewed from the laminating direction. In other words, the end portion 21d in the longitudinal direction A is positioned more on the outside than the end portion 11d of the first strip member 1. Furthermore, a tip end of the end portion 21d in the longitudinal direction A is positioned more on the outside than the second bonding portion 6. In other words, the tip end of the end portion 21d protrudes from the second bonding portion 6.

As illustrated in FIG. 2(a), a plurality of penetrating portions 23 along the laminating direction are provided on a portion of the substrate layer 21. The penetrating portions 23 are provided on a portion not configuring the first end portion 2a and second end portion 2b of the second strip member 2 on the substrate layer 21. The penetrating portions 23 are provided such that a portion of an edge along the longitudinal direction A of the substrate layer 21 is recessed. The penetrating portions 23 are provided by performing a notching, piercing, or the like on a portion of the substrate layer 21. In a case where the securing member 10 is in the laminated condition by providing the penetrating portions 23, a portion of the adhesive layer 12 of the strip member 1 can contact the third strip member 3 through the penetrating portions 23. Note that only one of the penetrating portions 23 may be provided on the substrate layer 21.

The substrate layer 21 is configured from a resin film for example. The substrate layer 21 may have a single layer structure or may have a multilayer structure. The substrate layer 21 has different stretching properties than the substrate layer 11. In the first embodiment, the substrate layer 21 has elasticity, and a width of at least a portion thereof narrows when a tensile load is applied in the longitudinal direction A. Therefore, in a case where the substrate layer 21 stretches along the longitudinal direction A, the second strip member 2 provided with the substrate layer 21 elastically deforms as illustrated in FIG. 4. At this time, a portion of the substrate layer 21 with a width that narrows by providing the penetrating portions 23 is prone to extending more than another portion.

An elastomer is included in the resin material configuring the substrate layer 21. The type of elastomer is not particularly limited, and examples at least include styrene elastomers and olefin elastomers. More specific examples of the elastomers include styrene-isoprene-styrene block copolymers (SIS), styrene butadiene-styrene block copolymers (SBS), styrene-ethylene/butylene-styrene block copolymers (SEBS), polyurethanes, polyolefins, (ethylene copolymers (such as ethylene vinyl acetates, ethylene-propylene copolymers, and ethylene-propylene-diene terpolymers), polypropylene copolymers, and polypropylene homopolymers), and the like. Furthermore, the resin material configuring the substrate layer providing stretching properties may include the elastomer and another component. For example, the resin material may include a stiffening agent (such as polyvinyl styrene, polystyrene, poly α-methyl styrene, polyester, epoxy resin, polyolefin, and coumarone-indene resin), viscosity reducing agent, plasticizer (such as an aliphatic hydrocarbon tackifier, aromatic hydrocarbon tackifier, terpene resin tackifier, and hydrogenated terpene resin tackifier), dye, pigment, antioxidant, antistatic agent, adhesive, antitack agent, slipping agent, heat stabilizer, light stabilizer, foaming agent, glass bubbles, starch, metal salt, microfiber, or the like.

A load when the substrate layer 21 is stretched 200% from an initial condition along the longitudinal direction A is 0.5 N/25 mm or greater or 1 N/25 mm or greater, and 20 N/25 mm or less or 17 N/25 mm or less for example. When the load is 0.5 N/25 mm, the substrate layer 21 can be prevented from immediately stretching when the first strip member 1 and second strip member 2 are opened. When the load is 20 N/25 mm or less, the substrate layer 21 can be stretched by an appropriate force. Note that the substrate layer 21 being stretched 200% along the longitudinal direction A indicates that the long side length of the substrate layer 21 after stretching is 300% with regard to the long side length (initial long side length) of the substrate layer 21 before stretching.

After the substrate layer 21 is stretched 300% along the longitudinal direction A, a load when returned to 200% is 0.1 N/25 mm or greater or 0.2 N/25 mm or greater, and 10 N/25 mm or less or 5 N/25 mm or less, for example. When the load is 0.1 N/25 mm or greater, a winding force can be favorably applied by the substrate layer 21 when the securing member 10 in the opened condition is wound on the member to be secured. When the load is 10 N/25 mm or less, a winding force by the substrate layer 21 can be appropriate when the securing member 10 in the opened condition is wound on the member to be secured.

After the substrate layer 21 is stretched 300% along the longitudinal direction A, a residual strain when the load of the substrate layer 21 after stretching (return load) is 0.1 N/25 mm or less is 1% or higher or 5% or higher, and 180% or lower or 160% or lower for example. When the residual strain is 1% or higher, a winding force can be favorably applied by the substrate layer 21 when the securing member 10 in the opened condition is wound on the member to be secured. When the residual strain is 180% or lower, a winding force by the substrate layer 21 can be appropriate when the securing member 10 in the opened condition is wound on the member to be secured.

When the substrate layer 21 is stretched 300% along the longitudinal direction A, the degree of reduction of the short side length of the substrate layer 21 (in other words, the degree of reduction of a width W1 of the substrate layer 21 illustrated in FIG. 4) is 20% or higher or 25% or higher, and 60% or lower or 50% or lower, for example. When the degree of reduction is 20% or higher, a pressure-sensitive adhesive force of the adhesive layer 22 provided above the surface 21a of the substrate layer 21 can be reduced in the securing member 10 in the opened condition. When the degree of reduction is 60% or lower, the stretched substrate layer 21 can be prevented from being too thin and thus the substrate layer 21 can be prevented from breaking. Note that the width W1 of the substrate layer 21 defines a width at a narrowest location of the width of the stretched substrate layer 21. Furthermore, the degree of reduction of the width indicates a ratio of width shrinking due to stretching (value obtained by subtracting a minimum width during stretching from a width before stretching) with regard to the width of the substrate layer 21 before stretching.

The load, residual strain, and degree of reduction can be measured in accordance with JIS K 7127:1995 (ISO 527-3:1995). For example, a 25 mm×75 mm test piece is prepared. Next, both end portions in a long side direction of the test piece are covered by a tape (for example, Filament Tape #898 manufactured by 3M Japan Ltd.) such that an area of the test piece on a pulled portion is 25 mm×25 mm. Furthermore, the test piece is stretched along the longitudinal direction under a 300 mm/min stretching rate condition, using a tensile tester or a device having a similar ability thereto. Thereby, the load, residual strain, and degree of reduction are measured.

The substrate layer 21 having the aforementioned properties is used, and therefore, a maximum long side length of the securing member 10 when the second strip member 2 including the substrate layer 21 is stretched can be at least 3.5 times or more (250% or greater) the long side length of the securing member 10 in the laminated condition. For example, when the long side length of the securing member 10 in the laminated condition is set to 45 mm, a maximum long side length of the securing member 10 in the opened condition can be 160 mm or greater. Thereby, the dimensions (and particularly the long side length of the securing member 10) in the laminated condition of the securing member 10 provided with the substrate layer 21 before stretching can be made compact.

The adhesive layer 22 is a layer maintaining the laminated condition of the securing member 10 similar to the adhesive layer 12, and the adhesive layer 22 is detachably adhered to a substrate layer 31 (described later in detail) of the third strip member 3, and adhered to an adhesive layer 32 (described later in detail) of the third strip member 3 by the second bonding portion 6. Therefore, in a case where the securing member 10 is in a laminated condition, the second strip member 2 and third strip member 3 are in close contact, and a laminated condition thereof is maintained. The adhesive force of the second strip member 2 and third strip member 3 (in other words, adhesive force when the adhesive layer 22 is adhered to the substrate layer 31) is 0.1 N/25 mm or greater or 0.3 N/25 mm, and 10 N/25 mm or less or 5 N/25 mm. When the adhesive force is 0.1 N/25 mm or greater, the second strip member 2 can be prevented from inadvertently opening from the third strip member 3. When the adhesive force is 10 N/25 mm or less, the second strip member 2 can be detached from the third strip member 3 by an appropriate force. A thickness of the adhesive layer 22 is from 5 μm to 100 μm, for example.

A portion provided on the end portion 21c of the substrate layer 21 on the adhesive layer 22 configures the first end portion 2a of the second strip member 2. Furthermore, a portion of the adhesive layer 22 on the end portion 21c faces a first strip member 1 side and is directly adhered to the adhesive layer 12. Therefore, the first bonding portion 5 of the first strip member 1 and first end portion 2a of the second strip member 2 are firmly adhered to each other by the adhesive layers 12, 22. Therefore, even when the securing member 10 is opened, the first strip member 1 and second strip member 2 are integrated by the first bonding 5, and can be favorably suppressed from separating. Furthermore, a portion provided on the end portion 21d of the substrate layer 21 on the adhesive layer 22 configures the second end portion 2b of the second strip member 2.

The adhesive layer 22 is formed by coating a pressure-sensitive adhesive or the like on the surface 21a of the substrate layer 21. A pressure-sensitive adhesive included in the adhesive layer 22 is the same as the pressure-sensitive adhesive configuring the adhesive layer 12.

The third strip member 3 is a portion secured in advance to the member to be secured on the securing member 10. Furthermore, the third strip member 3 is a portion which is a second end in the longitudinal direction A for the opened securing member 10. The third strip member 3 has the substrate layer 31 (third substrate layer) and adhesive layer 32 (third adhesive layer).

The substrate layer 31 is a portion having a strip shape, and has a surface 31 a (first surface) which is a surface on a side opposite from the outermost layer 15, and a surface 31b (second surface) facing a second strip member 2 side and first strip member 1 side, on a side opposite from the surface 31a. The adhesive layer 32 is provided above the surface 31a. The second strip member 2 and first strip member 1 are laminated in order above the surface 31b. At least a portion of the adhesive layer 22 of the second strip member 2 is detachably adhered to the surface 31b. Furthermore, as described above, a portion of the adhesive layer 12 of the first strip member 1 is detachably adhered through the penetrating portions 23 to the surface 31b. As viewed from the laminating direction, a long side length of the substrate layer 31 is from 5 mm to 100 mm, and a short side length of the substrate layer 31 is essentially identical at from 2 mm to 30 mm. A thickness of the substrate layer 31 is from 20 μm to 200 μm, for example.

The first end portion 31c of the substrate layer 31 in the longitudinal direction A is exposed from the first strip member 1 and third strip member 3 as viewed from the laminating direction, and is covered by the second strip member 2. Furthermore, the end portion 31d of the substrate layer 31 in the longitudinal direction A is a portion configuring the second bonding portion 6. When the securing member 10 is in the laminated condition, the end portion 31d is folded toward the second strip member 2 side. Therefore, a portion of the surface 31a faces the second strip member 2 side on the end portion 31d.

The substrate layer 31 is configured from a resin film, paper, or nonwoven material for example. A resin configuring the substrate layer 31 is the same as the resin configuring the substrate layer 11.

The adhesive layer 32 is a layer that adheres the substrate layer 31 and member to be secured. A portion of the portion provided on the end portion 31d of the substrate layer 31 on the adhesive layer 32 faces a second strip member 2 side and is directly adhered to the adhesive layer 22. Therefore, the second bonding portion 6 of the third strip member 3 and second end portion 2b of the second strip member 2 are firmly adhered to each other by the adhesive layers 22, 32. Therefore, even when the securing member 10 is opened, the second strip member 2 and third strip member 3 are integrated by the second bonding 6, and can be favorably suppressed from separating. A thickness of the adhesive layer 32 is from 5 μm to 100 μm, for example. The adhesive layer 32 is formed by coating a pressure-sensitive adhesive or the like on the surface 31a of the substrate layer 31. A pressure-sensitive adhesive included in the adhesive layer 32 is the same as the pressure-sensitive adhesive configuring the adhesive layers 12, 22.

FIG. 5 is a view illustrating a sheet as a base for the securing member 10 according to the first embodiment. As illustrated in FIG. 5, a sheet 5 which is a base for the securing member 10 is a strip-shaped laminate body attached to an unwinding device or the like in a condition wound on a roller 50. A winding method of the sheet 51 to the roller 50 may be a winding method where a winding direction and rotational direction are parallel as with so-called toilet paper (parallel winding), or a winding method where the winding direction and rotational direction are oblique as with a bobbin (bobbing winding). A length of the sheet 51 along an unwinding direction of the roller 50 is 100 m or greater, for example. On the sheet 51 wound on the roller 50, an individual portion cut along a direction orthogonal to an extending direction corresponds to the securing member 10. Herein, as described above, the first bonding portion 5 is positioned on a first end side of the securing member 10, the second bonding portion 6 is positioned on a second end side of the securing member 10, and the tab portion 13 and latching portion 14 are provided between the first bonding portion 5 and second bonding portion 6. Furthermore, the first bonding portion 5, second bonding portion 6, tab portion 13, and latching portion 14 are provided so as to not mutually overlap in the laminating direction. Therefore, a specific location is less likely to become thick on the sheet 51 wound on the roller 50, and therefore, a sheet 51 with favorable quality can be formed.

In FIG. 5, when the sheet 51 is wound on the roller 50, a portion which later becomes the adhesive layer 32 of the securing member 10 is adhered to a portion which later becomes the outermost layer 15 of the securing member 10. At this time, when the surface of the outermost layer 15 is an uneven surface, peeling of a portion which later becomes the adhesive layer 32 of the securing member 10 and a portion which later becomes the outermost layer 15 of the securing member 20 will be easier. Therefore, the sheet 51 can smoothly unwind from the roller 50.

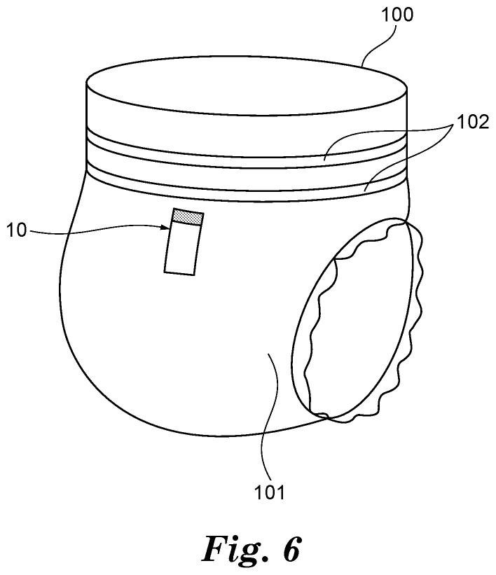

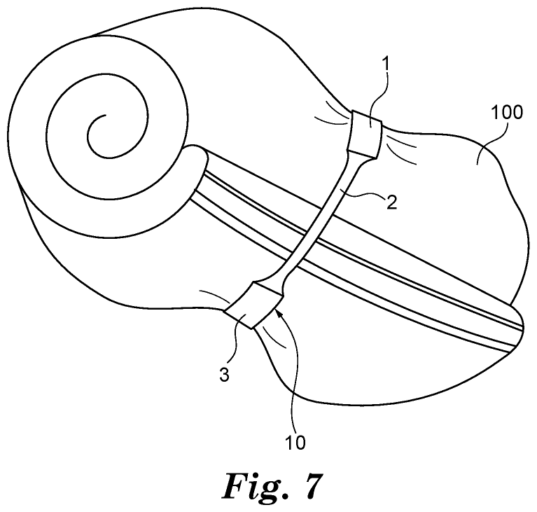

Next, a function and effect when securing the member to be secured using the securing member 10 according to the first embodiment will be described while referring to FIG. 6 and FIG. 7. FIG. 6 is a schematic view illustrating a condition where the securing member in the laminated condition is adhered to the member to be secured. FIG. 7 is a view illustrating a condition where the securing member in the opened condition is wound and secured on the member to be secured.

As illustrated in FIG. 6, a disposable diaper 100 serving as a member to be secured is provided with a main body portion 101 that internally provides an absorbent body and where a pair of leg opening portions are provided, and a waist portion 102 provided on an upper side of the main body portion 101, that provides a waist opening portion, and that suppresses deviation when a wearer wears the disposable diaper 100. The main body portion 101 is configured from paper, a nonwoven material, and resin film for example. A design or the like may be printed on an outer surface of the main body portion 101.

When the securing member 10 is in the laminated condition, the adhesive layer 32 exposed by the securing member 10 (refer to FIG. 1(b)) is attached to the main body portion 101 of the disposable diaper 100 serving as the member to be secured. At this time, reduction in the dimensions of the securing member 10 (and particularly the long side length of the securing member 10) is achieved, and therefore, adhering to the main body portion 101 so as to not overlap the waist portion 102 is possible. Thereby, the securing member 10 is less likely to contact the wearer of the disposable diaper 100, and thus discomfort for the wearer can be reduced. Furthermore, the securing member 10 in the laminated condition has the aforementioned dimensions, and therefore, a design or the like printed on an outer surface of the main body portion 101 is less likely to be hidden by the securing member 10. Therefore, the aesthetic appearance of the disposable diaper 100 can be suppressed from being impaired by the securing member 10.

When the disposable diaper 100 is used and disposed, the contents inside the disposable diaper 100 must be prevented from leaking to the outside. Therefore, as illustrated in FIG. 7, a condition where the disposable diaper 100 is rounded and assembled is maintained by the securing member 10, and therefore, leak prevention of the contents is achieved. Specifically, the tab portion 13 is gripped and pulled in the laminating direction, and therefore, the adhesive layer 12 is detached from the substrate layers 21, 31 and the adhesive layer 22 is detached from the substrate layer 31. Next, the securing member 10 is wound on the disposable diaper 100 with the first strip member 1 as a starting point. Furthermore, the adhesive layer 12 is adhered to the disposable diaper 100 in a condition where the securing member 10 is wound on the disposable diaper 100. Thereby, a condition where the disposable diaper 100 is assembled by the securing member 10 can be favorably maintained. At this time, a tensile load is applied to the securing member 10 along the longitudinal direction A to elastically deform the substrate layer 21, and therefore, the securing member 10 can be firmly wound on the disposable diaper 100 in a condition where the substrate layer 21 is stretched.

Herein, for the securing member 10, when a crease on the second bonding portion 6 is undone when changing from the laminated condition to the opened condition, the orientation of the second strip member 2 is reversed. In other words, the surface 21a of the substrate layer 21 in the laminated condition and the surface 21a of the substrate layer 21 in the opened condition face mutually opposite sides in the laminating direction (refer to FIG. 1(b) and FIG. 2(b)). Therefore, when the securing member 10 is secured to the member to be secured (when the adhesive layers 12, 32 are adhered to the disposable diaper 100 in FIG. 7), the adhesive layer 22 does not structurally adhere to the disposable diaper 100, and is exposed to an outer side. Thereby, the adhesive layer 22 may adhere to a wearer assembling the disposable diaper 100 or to another article such as disposal bag or the like. In this case, the assembled disposable diaper 100 needs to be handled such that the adhesive layer 22 does not adhere to another article or the like. In contrast, in the first embodiment, when the securing member 10 is wound on the disposable diaper 100, a tensile load is applied on the securing member 10 along the longitudinal direction A to elastically deform the substrate layer 21, and therefore, a condition is achieved where the width of at least a portion of the substrate layer 21 is narrow. Thereby, the adhesive layer 22 is compressed, and therefore, at least a portion is wrapped in the substrate layer 21, and thus the adhesive force of the adhesive layer 22 reduced. Therefore, even when the adhesive layer 22 is not adhered to the disposable diaper 100, the securing member 10 can be suppressed from adhering to an article other than the disposable diaper 100.

Furthermore, when the securing member 10 is wound on the disposable diaper 100 in a condition where the width of at least a portion of the substrate layer 21 is narrow, a winding force applied by the substrate layer 21 on the disposable diaper 100 increases per unit length. Thereby, as illustrated in FIG. 7, the substrate layer 21 easily bites into the disposable diaper 100, and the adhesive layer 22 easily adheres to a portion not wound on the securing member 10 on the disposable diaper 100. Alternatively, the adhesive layer 22 is less likely to contact an article or the like other than the disposable diaper 100. Therefore, in a case where the member to be secured is an article have flexibility as with the disposable diaper 100, the securing member 10 can be favorably suppressed from adhering to an article other than the disposable diaper 100.

In the first embodiment, when the substrate layer 21 is stretched 300% along the longitudinal direction A, the degree of reduction of the width of the substrate layer 21 may be 20% to 60%. In this case, when the degree of reduction is 20% or higher, a pressure-sensitive adhesive force of the adhesive layer 22 provided above the surface 21a of the substrate layer 21 can be reduced in the securing member 10 in the opened condition. When the degree of reduction is 60% or lower, the stretched substrate layer 21 can be prevented from being too thin and thus the substrate layer 21 can be prevented from breaking.

In the first embodiment, the load when the substrate layer 21 is stretched 200% along the longitudinal direction A may be from 0.5 N/25 mm to 20 N/25 mm. In this case, when the load is 0.5 N/25 mm, the substrate layer 21 can be prevented from immediately stretching when the first strip member 1 and second strip member 2 are opened. Furthermore, when the load is 20 N/25 mm or less, the substrate layer 21 can be stretched by an appropriate force.

In the first embodiment, the load when the substrate layer 21 is stretched 300% along the longitudinal direction A and then returned to 200% may be from 0.1 N/25 mm to 10 N/25 mm. When the load is 0.1 N/25 mm or greater, a winding force can be favorably applied by the substrate layer 21 when the securing member 10 in the opened condition is wound on the member to be secured. When the load is 10 N/25 mm or less, a winding force by the substrate layer 21 can be appropriate when the securing member 10 in the opened condition is wound on the member to be secured.

In the first embodiment, the penetrating portions 23 are provided along the laminating direction on the substrate layer 21, and the adhesive layer 12 may be detachably adhered to the surface 31a of the substrate layer 31 through the penetrating portion 23. In this case, the laminated condition of the securing member 10 before use is favorably maintained by the adhesive layer 12. Herein, the adhesive force of the adhesive layer 12 is preferably stronger than the adhesive force of the adhesive layer 22. Thereby, the laminated condition of the securing member 10 before use is more favorably maintained by the adhesive layer 12.

In the first embodiment, the first strip member 1 may be provided above the surface 11a of the substrate layer 11, and have the latching portion 14 that latches to the member to be secured. In this case, adhering of the member to be secured by the first strip member 1 is achieved by both the adhesive layer 12 and latching portion 14. Therefore, when the area of the adhesive layer 12 is reduced due to a reduction in size, a latching force of the first strip member 1 can be provided without increasing the adhesive force of the adhesive layer 12. When the adhesive force of the adhesive layer 12 is too high, there are problems where opening from the laminated condition is difficult and the like, and therefore, occurrences of the aforementioned problems can be prevented by using the latching portion 14. Furthermore, in a case where the member to be secured is the aforementioned diaper or the like, when assuming that moisture such as urine or the like is included in the member to be secured, the adhesive force of the adhesive layer 12 may weaken due to the moisture. Even in this case, adhering of the first strip member 1 and member to be secured is maintained by the latching portion 14. Therefore, adhering of the member to be secured by the first strip member 1 can be more reliable. Furthermore, in a case where the substrate layer 21 elastically deforms, resilience of the substrate layer 21 is provided on the latching portion 14. Thereby, latching of the member to be secured by the latching portion 14 is firm.

In the first embodiment, the securing member 10 may have the tab portion 13 positioned on a second end portion (in other words, end portion 11d of the substrate layer 11) on a side opposite from the first end portion configuring the first bonding portion 5 on the first strip member 1, and the latching portion 14 may be disposed so as to not overlap with the tab portion 13, first bonding portion 5, and second bonding portion 6 in the laminating direction when the securing member 10 is in the laminated condition. In this case, a specific location of the securing member 10 in the laminated condition is less likely to be thick, and therefore, the quality of the sheet 51 wound on the roller 50 as illustrated in FIG. 5 can be improved, for example.

In the first embodiment, the securing member 10 may have the tab portion 13 positioned on a second end portion (in other words, end portion 11d of the substrate layer 11) on a side opposite from the first end portion configuring the first bonding portion 5 on the first strip member 1, and the latching portion 14 may be disposed between the tab portion 13 and first bonding portion 5 in the longitudinal direction A with 3 mm to 30 mm intervals from an end portion on the first bonding portion 5 side of the tab portion 13. In this case, the adhesive force of the adhesive layer 12 in the laminated condition is sufficiently maintained. Furthermore, adhesion of the adhesive layer 12 positioned between the tab portion 13 and latching portion 14 is sufficiently demonstrated, and the end portion 11d of the adhesive layer 12 can be suppressed from easily peeling from the second strip member 2.

In the first embodiment, the securing member 10 may be latched to the member to be secured at a 90 degree peeling force of 0.5 N/25 mm or greater by the latching portion 14 and adhesive layer 12. In this case, adhering of the first strip member 1 to the member to be secured is sufficiently maintained.

In the first embodiment, the securing member 10 may be latched to the member to be secured at a dynamic shear force of 4 N/(25 mm×25 mm) or greater by the latching portion 14 and adhesive layer 12. In this case, adhering of the first strip member 1 to the member to be secured is sufficiently maintained.

In the first embodiment, the long side length of the securing member 10 in the laminated condition may be 5 mm to 50 mm.

In the first embodiment, the aforementioned disposable diaper 100 may be used as the member to be secured, for example. Furthermore, after 200 mL of 40° C. water is absorbed by the disposable diaper 100 where the securing member 10 (specifically, adhesive layer 32) is adhered and then left to stand for two minutes, the opened securing member 10 is wound while pulling at a force of 500 gf. Next, after leaving to stand for one hour in a condition where the adhesive layer 12 is adhered to the disposable diaper 100, the adhered condition of the adhesive layer 12 may be maintained. In this case, even with the aforementioned member to be secured, the securing member 10 can maintain for a long period of time a condition where the member to be secured is assembled.

In the first embodiment, a tip end of the end portion 21d of the substrate layer 21 may protrude from the second bonding portion 6. In this case, when the securing member 10 is opened, the adhesive layer 32 of the third strip member 3 is adhered to the tip end of the substrate layer 21. Thereby, integration of the second strip member 2 and third strip member 3 can be firmer.

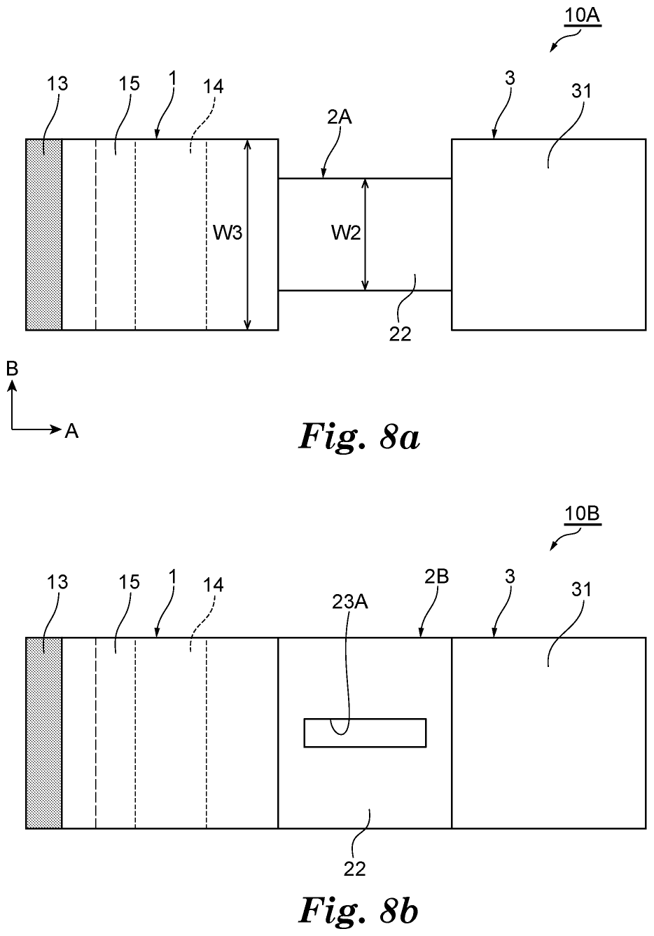

FIG. 8(a) is a view illustrating an opened condition of a securing member according to a first modified example of the first embodiment. As illustrated in FIG. 8(a), a width W2 of the second strip member 2A of a securing member 10A according to the first modified example is shorter than a width W3 of the first strip member 1 (and width of the third strip member 3). For example, the width W2 is from 30% to 95% of the width W3. In this case, the adhesive layer 12 can be favorably adhered to the surface 31b, and the second strip member 2A can have a sufficient breaking resistance. Even in this case, when the securing member 10A is in a laminated condition, at least a portion of the adhesive layer 12 of the first strip member 1 does not overlap with a second strip member 2A, and directly adheres to the surface 31b of the substrate layer 31 on the third strip member 3 (refer to FIG. 1(b)). Therefore, according to the first modified example, the laminated condition of the first strip member 1, second strip member 2, and third strip member 3 is maintained by the adhesive layer 12 similar to the first embodiment, and therefore, the laminated condition of the securing member 10A before use is favorably maintained.

FIG. 8(b) is a view illustrating an opened condition of a securing member according to a second modified example of the first embodiment. As illustrated in FIG. 8(b), an opening portion 23A serving as a penetrating portion is provided on a second strip member 2B of a securing member 10B according to the second modified example. Even in this case, when the securing member 10A is in a laminated condition, at least a portion of the adhesive layer 12 of the first strip member 1 directly adheres to the surface 31b of the substrate layer 31 on the third strip member 3 through the opening portion 23A (refer to FIG. 1(b)). Therefore, according to the second modified example, a laminated condition of the securing member 10B before used is favorably maintained by the adhesive layer 12 similar to the first embodiment. Note that a plurality of opening portions may be provided on the second strip member 2B. Furthermore, the shape of the opening portion is not particularly limited, and may be a circular shape as view from the laminating direction, an elliptical shape, or polygonal shape.

### Second Embodiment

A securing member according to a second embodiment will be described below. Duplicate descriptions from the first embodiment are omitted in the description of the second embodiment, and different portions from the first embodiment are described. In other words, in a technically possible range, a description of the first embodiment may be appropriately used in the second embodiment.

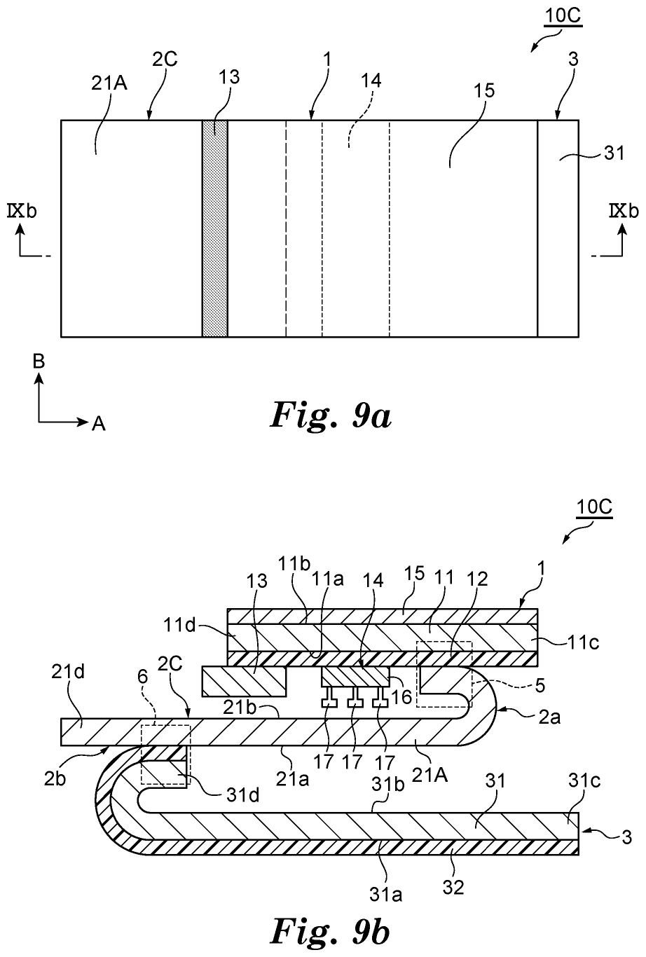

FIG. 9(a) is a plan view of a securing member according to a second embodiment, and FIG. 9(b) is a view illustrating a cross section along line IXb-IXb in FIG. 9(a). As illustrated in FIG. 9(b), a second strip member 2C of a securing member 10C only has a substrate layer 21A. In other words, the second strip member 2C is different from the first embodiment and is provided with the adhesive layer 22.

The substrate layer 21A is different from the substrate layer 21 in the first embodiment, and is a layer that does not have flexibility but can deform by applying a tensile load. Specifically, the substrate layer 21A is a layer where plastic deformation is possible based on applying a tensile load. Furthermore, the substrate layer 21A is provided with the penetrating portions similar to the first embodiment (refer to FIG. 2(a)). In this case, a portion of the substrate layer 21A with a width that narrows by providing the penetrating portions is prone to plastic deformation more than another portion.

When the securing member 10C according to the second embodiment is in a laminated condition, the adhesive layer 12 is adhered to the surface 21b of the substrate layer 21A, and is adhered to the surface 31b of the substrate 31 through the penetrating portions. Therefore, the laminated condition of the securing member 10C is favorably maintained by the adhesive layer 12. Furthermore, when the securing member 10C is in an opened condition, the substrate layer 21A plastic deforms, and therefore, the securing member 10C can be favorably wound on the member to be secured. Furthermore, the second strip member 2 is not provided with an adhesive layer. Therefore, when the adhesive layers 12, 32 of the securing member 10C in the opened condition is adhered to the member to be secured, a protruding adhesive layer is not present on the securing member 10C. Therefore, the securing member 10C can be favorably prevented from adhering to another article or the like.

Note that the substrate layer 21A does not have the aforementioned penetrating portions, and may have a narrower width than the first strip member 1 similar to the first modified example of the first embodiment (refer to FIG. 8(a)), and the opening portion 23A may be provided similar to the second modified example of the first embodiment (refer to FIG. 8(b)). Even in these cases, a function and effect according to the second embodiment are similarly achieved.

### Third Embodiment

A securing member according to a third embodiment will be described below. Duplicate descriptions from the first embodiment and second embodiment are omitted in the description of the third embodiment, and different portions from the first embodiment and second embodiment are described. In other words, in a technically possible range, a description of the first embodiment and second embodiment may be appropriately used in the third embodiment.

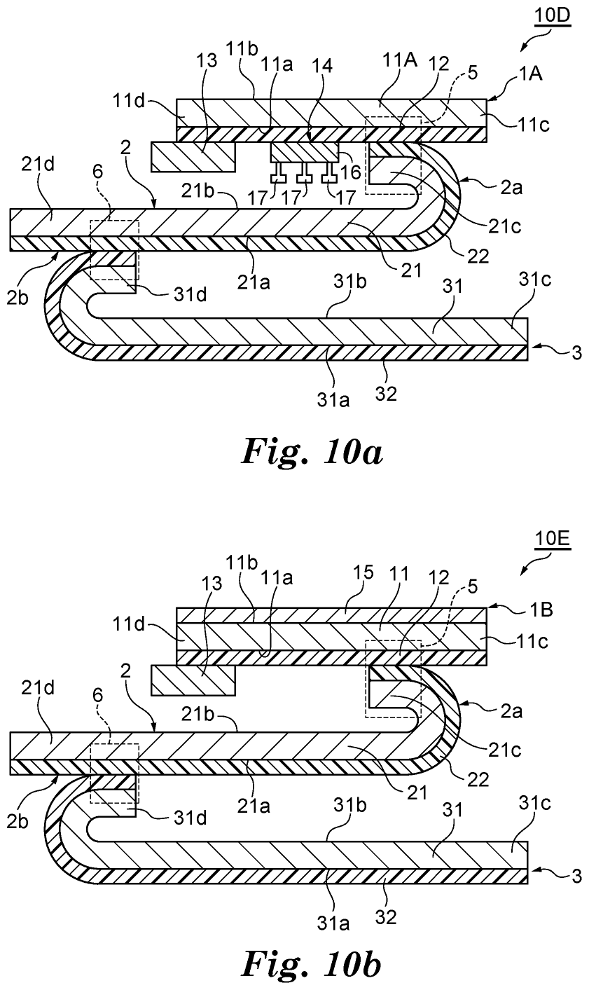

FIG. 10(a) is a view illustrating a cross of a securing member according to the third embodiment. A first strip member 1A of a securing member 10D illustrated in FIG. 10(a) is different from the first embodiment and second embodiment, and is not provided with the outermost layer 15. Therefore, the surface 11b of a substrate layer 11A is an outermost surface on the securing member 10D in a laminated condition. An uneven surface may be provided by an embossing process or the like on the substrate layer 11A of the third embodiment, for example. With the securing member 10D according to the third embodiment, the surface 11b of the substrate layer 11A is directly made into an uneven surface, and therefore, further reduction in cost, man-hours, and the like can be achieved while providing integral appearance and feel, as compared to a case where the uneven outermost layer 15 is provided.

### Fourth Embodiment

A securing member according to a fourth embodiment will be described below. Duplicate descriptions from the first embodiment to third embodiment are omitted in the description of the fourth embodiment, and different portions from the first embodiment to third embodiment are described. In other words, in a technically possible range, a description of the first embodiment to third embodiment may be appropriately used in the fourth embodiment.

FIG. 10(b) is a view illustrating a cross of a securing member according to the fourth embodiment. A first strip member 1B of the securing member 10E illustrated in FIG. 10(b) is different from the first embodiment to third embodiment, and does not provide the latching portion 14. With the securing member 10D according to the third embodiment, an area of the adhesive layer 12 attached to the member to be secured in the opened condition can be increased, and thus a function and effect similar to the first embodiment and third embodiment are achieved.

### Fifth Embodiment

A securing member according to a fifth embodiment will be described below. Duplicate descriptions from the first embodiment to fourth embodiment are omitted in the description of the fifth embodiment, and different portions from the first embodiment to fourth embodiment are described. In other words, in a technically possible range, a description of the first embodiment to fourth embodiment may be appropriately used in the fifth embodiment.

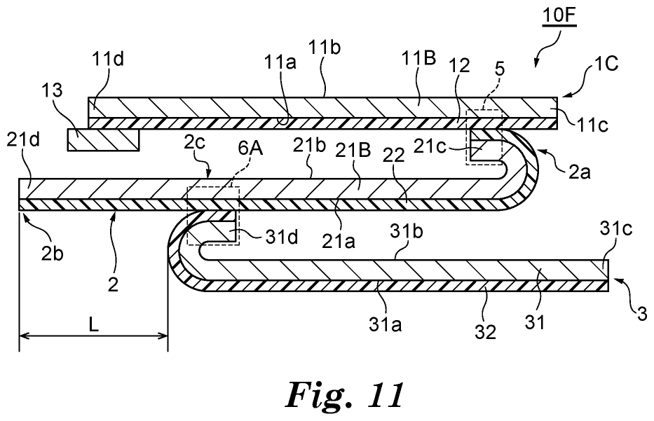

FIG. 11 is a view illustrating a cross of a securing member according to the fifth embodiment. For a securing member 10F illustrated in FIG. 11, the long side length of the first strip member and second strip member is different as compared to the third embodiment, for example. Specifically, the end portion 11d of the substrate layer 11B on a first strip member 1C is positioned more on the outside than the second bonding portion 6 in the longitudinal direction A. Furthermore, the end portion 11d is provided so as not to overlay with the first bonding portion 5 and second bonding portion 6 in the laminating direction. Furthermore, tab portion 13 positioned on the end portion 11d of the substrate layer 11B is also positioned more on the outside than the second bonding portion 6 in the longitudinal direction A, and is provided so as not to overlay with the first bonding portion 5 and second bonding portion 6 in the laminating direction.

The end portion 21d of the substrate layer 21B is positioned more on the outside than the second bonding portion 6 in the longitudinal direction A. Therefore, the second bonding portion 6 is configured from a portion 2c more on a center side than the second end portion 2b on a second strip member 2D, and the third strip member 3. On the second strip member 2D, the portion 2c is positioned between the first end portion 2a and second end portion 2b on a second end portion 2b. Furthermore, the end portion 21d of the substrate layer 21B is positioned more on the outside than the tab portion 13 and end portion 11d of the substrate layer B in the longitudinal direction A. In other words, the end portion 21d is a first end in the longitudinal direction A of the securing member 10F in the laminated condition. A length L of the substrate layer 21B from the second bonding portion 6A to a tip end of the second end portion 2b of the second strip member 2D in the longitudinal direction A is larger than a protruding amount of the end portion 21d in the first embodiment, for example. For example, the length L is from 3 mm to 20 mm.

A similar function and effect to the third embodiment is also achieved with the securing member 10F according to the fifth embodiment. Furthermore, as described above, the length L of the substrate layer 21B in the fifth embodiment is larger than a protruding amount of the end portion 21d in the first embodiment. Therefore, when the securing member 10F is opened after adhering the adhesive layer 32 to the member to be secured, the end portion 21d of the substrate layer 21B can also be adhered to the member to be secured along with the adhesive layer 32, and thus the third strip member 3 side of the securing member 10F is less likely to peel from the member to be secured.

The present invention was described in detail above based on the aforementioned embodiments and modified examples. However, the present invention is not limited to the aforementioned embodiments and modified examples. The present invention can be further modified within a scope that does not depart from the gist thereof.

For example, the aforementioned embodiments and modified examples may be appropriately combined. For example, the contents of the first modified example of the first embodiment or the contents of the second modified example may be combined with the second embodiment. Furthermore, the contents of the second embodiment may be combined with the third embodiment or fourth embodiment. Furthermore, the contents of the fifth embodiment may be combined with another embodiment or modified example.

For example, an adhesive layer may not be provided on the second strip member in the aforementioned first embodiment, third embodiment, and fourth embodiment.

For example, the penetrating portions 23 and opening portion 23A may not be provided on the second strip member in the aforementioned first embodiment, third embodiment, and fourth embodiment. In this case, the laminated condition of the first strip member and second strip member is maintained only by the adhesive layer 12, and the laminated condition of the second strip member and third strip member is maintained only by the adhesive layer 22.

### EXAMPLES

The present invention will be further described in detail by the following Examples, but the present invention is not limited to these examples.

### Example 1

In Example 1, a securing member having a cross-sectional shape as illustrated in FIG. 10 of the aforementioned embodiment is formed. Specifically, a first strip member and a first end portion of a second strip member were adhered, and a third strip member and a second end portion of the second strip member were adhered, and therefore, the first strip member, second strip member, and third strip member were arranged in a linear shape to first form an integral member. Furthermore, the first end portion of the second strip member was folded back and a first portion of the third strip member was folded, and thus the first to third strip members were folded and overlaid to form a securing member. A long side length of the first strip member was 32 mm, a long side length of the second strip member was 44 mm, and a long side length of the third strip member was 49 mm. As described above, a portion of the second strip member and third strip member were folded back, and therefore, a long side length of the securing member was 45 mm.

The first strip member is provided with an 80 μm thick polypropylene film (first substrate layer), a layer-shaped pressure-sensitive adhesive (first adhesive layer), a tab portion including a 80 μm thick polypropylene film, and a hook member with 1600 pins/square inch (product name: 1600 DH available from 3M Japan Ltd.). In Example 1, the pressure-sensitive adhesive configuring the first adhesive layer is a styrene-isoprene-styrene pressure-sensitive adhesive with “Quintac 3520” (available from Zeon Corporation) as a base.

In the first strip member, a length of the hook member along a longitudinal direction of the securing member was 9 mm, and a distance along the longitudinal direction between the hook member and tab portion was 7 mm. Furthermore, a distance along the longitudinal direction between the hook member and a point where the first strip member and second strip member contact in a laminated condition was 8 mm.

The second strip member is provided with a film (second substrate layer, thickness: 80 μm) having at least one styrene-isoprene-styrene elastomer as a resin material, and a layer-shaped pressure-sensitive adhesive (second adhesive layer). The pressure-sensitive adhesive configuring the second adhesive layer is a styrene-isoprene-styrene pressure-sensitive adhesive with “D107 JSP” (available from Kraton Polymer Japan KK) as a base. Note that the adhesive force of the pressure-sensitive adhesive used in the second strip member is weaker than the adhesive force of the pressure-sensitive adhesive used in the first strip member.

The third strip member is provided with an 80 μm polypropylene film (third substrate layer), and a layer-shaped pressure-sensitive adhesive (third adhesive layer). The pressure-sensitive adhesive configuring the third adhesive layer is the same as the pressure-sensitive adhesive configuring the first adhesive layer.

### Example 2

In Example 2, other than a film (thickness: 120 μm) having at least one styrene-isoprene-styrene elastomer as a resin material was used as the second substrate layer of the second strip member, a securing member was formed similarly to Example 1.

### Example 3

In Example 3, other than a film (thickness: 80 μm) having at least one polyolefin elastomer as a resin material was used as the second substrate layer of the second strip member, a securing member was formed similarly to Example 1.

### Example 4

In Example 4, other than the first strip member did not have a hook member, a securing member was formed similarly to Example 1.

### Example 5

In Example 5, other than the first strip member did not have a hook member, a securing member was formed similarly to Example 2.

### Example 6

In Example 6, other than the first strip member did not have a hook member, a securing member was formed similarly to Example 3.

### Example 7

In Example 7, other than the pressure-sensitive adhesive used in the first strip member was the same as the pressure-sensitive adhesive used in the second strip member, a securing member was formed similarly to Example 1.

### Comparative Example 1

In Comparative Example 1, other than the first strip member did not have a hook member, the pressure-sensitive adhesive used in the first strip member was the same as the pressure-sensitive adhesive used in the second strip member, and the second substrate layer of the second strip member was a polypropylene film (thickness: 80 μm) without an elastomer, a securing member was formed similarly to Example 1.

### Stretching Properties of Second Substrate Layer

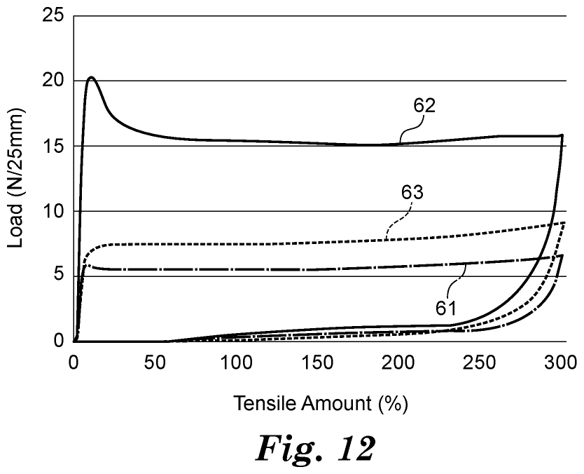

The stretching properties of the second substrate layer were measured in accordance with the measurement methods described in the aforementioned embodiments for Examples 1 to 3. The stretching properties include: a load (load a) when the second substrate layer is stretched 200% along the longitudinal direction A; a load (load b) when the second substrate layer is returned to 200% after stretching 300% along the longitudinal direction; a residual strain when a load (return load) of the second substrate layer after stretching is 0.1 N/25 mm or less after stretching the second substrate layer 300% along the longitudinal direction; and a degree of reduction of a width of the second substrate layer when the second substrate layer is stretched 300% along the longitudinal direction. The measurement results are shown in the following Table 1. Furthermore, FIG. 12 shows a hysteresis loop when the second substrate layer of Examples 1 to 3 is stretched and contracted in accordance with the aforementioned measurement methods. In FIG. 12, a graph 61 shows the results of Example 1, a graph 62 shows the results of Example 2, and a graph 63 shows the results of Example 3.

TABLE 1

| | Example 1 | Example 2 | Example 3 |
|---|---|---|---|
| Load a (N/25 mm) | 5.7 | 15.2 | 7.8 |
| Load b (N/25 mm) | 0.7 | 1.1 | 0.5 |
| Residual strain (%) | 74 | 66 | 144 |
| Degree of reduction (%) | 41 | 40 | 36 |

Peeling Force when Securing Member is Opened

An adhesive force (90 degree peeling force) between the first strip member and second strip member and an adhesive force (90 degree peeling force) between the second strip member and third strip member were measured in accordance with the measurement method described in the aforementioned embodiments for Examples 1 and 4. For both Examples 1 and 4, an adhesive force between the first strip member and second strip member was 2.5 N/mm, and an adhesive force between the second strip member and third strip member was 2.3 N/mm. Based on these results, the adhesive force was seen to be hardly affected even in a case where a hook member was provided on the first strip member.

Adhesive Force and Dynamic of First Strip Member with Regard to Nonwoven Material

For Examples 1 and 4 and Comparative Example 1, an adhesive force (90 degree peeling force) of the first strip member on a nonwoven material was measured in accordance with the measurement method described in the aforementioned embodiments. Furthermore, a dynamic shear strength of the first strip member with regard to a nonwoven material was measured in accordance with the measurement method of dynamic shear strength of a latching portion with regard to a nonwoven material described in the aforementioned embodiments. The nonwoven material was the same as the nonwoven material (PS-108-S available from Mitsui Chemicals, Inc.) described in the embodiments. The measurement results of Examples 1 and 4 and Comparative Example 1 are shown in the following Table 2.

TABLE 2

| | | | | Comparative |
|---|---|---|---|---|
| | Example 1 | Example 4 | Example 7 | Example 1 |
| Adhesive force | 2.5 | 4.2 | 1.4 | 1.2 |
| (N/25 mm) | | | | |
| Dynamic | 19.7 | 35.5 | 21.5 | 23 |
| shear strength | | | | |
| N/(25 mm × 25 mm)) | | | | |

Herein, in Example 1, measurements of the adhesive force and dynamic shear strength of only the hook member and measured of the adhesive force and dynamic shear strength of only the adhesive layer were performed. In a case where the adhesive force and dynamic shear strength of only the hook member were measured, a portion other than the hook member (in other words, adhesive layer) was masked by a tape (Masking tape for plating #851A available from 3M Japan Ltd.) for a test piece adhered to paper or a PET film. On the other hand, in a case where the adhesive force and dynamic shear strength of the adhesive layer were measured, a portion other than the adhesive layer (in other words, hook member) was masked by a tape for a test piece adhered to paper or a PET film. The measurement results are shown in the following Table 3.

TABLE 3

| | Hook member only | Adhesive layer only |
|---|---|---|
| Adhesive force (N/25 mm) | 1.2 | 1.3 |
| Dynamic shear strength | 4.8 | 13.1 |
| (N/(25 mm × 25 mm)) | | |

### Winding Test of Securing Member

For Examples 1 and 4 and Comparative Example 1, a winding test when a member to be secured is a commercially available diaper for children was performed by a method described below. First, a third strip member of a securing member with a 10 mm width (short side length) and 45 mm long side length was adhered to an outer surface of a main body portion of the diaper for children. Next, 200 mL of 40° C. lukewarm water was absorbed by the main body portion and then left to stand for two minutes. Next, the securing member was set to an opened condition and wound on the diaper for children while pulling at a force of 500 gf, and then the first strip member was adhered to a different outer surface of the main body portion. Furthermore, whether the first strip member peeled from the diaper for children after leaving to stand for one hour was visually observed.

The first strip member was observed to peel from the diaper for children for Examples 1, 4, and 7. In contrast, the first strip member was observed not to peel from the diaper for children for Comparative Example 1. This is considered to be because the pressure-sensitive adhesive used in the first strip member was weaker than Examples 1 and 3 and was weak to moisture absorbed by the diaper for children. On the other hand, for Example 7 using the same pressure-sensitive adhesive as Comparative Example 1, even in a case where an area of the pressure-sensitive adhesive protruding on the first strip member was reduced, winding on the diaper for children absorbing water was maintained.

For Example 1 and Comparative Example 1, a peeling force of the second strip member and a commercially available disposal bag for a diaper was measured in accordance with a measurement method described below. A disposal bag for a diaper in the present measurement method is used as an example of another article to which the protruding second adhesive layer adheres when the securing member is opened. First, a second strip member of securing member with a 25 mm width (short side length) and 50 mm long side length is prepared, both end portions on a long side were masked by a tape (filament tape #898 manufactured 3M Japan Ltd.) on a front and back of the sample, such that a protruding area of the sample is 25 mm×25 mm.

Next, each sample was set by the following three methods. (i) For the sample of Example 1, both ends in a long side direction were fixed by a tape on an aluminum panel such that an adhesive layer surface protrudes. (Hereinafter, referred to as “Example 1 before stretching”)

(ii) For the sample of Example 1, both ends in the long side direction were fixed by a tape on an aluminum panel such that an adhesive layer surface protrudes, while pulling in the long side direction at a force of 1000 gf. (Hereinafter, referred to as “Example 1 after stretching”)

(iii) For the sample of Comparative Example 1, both ends in the long side direction were fixed by a tape on an aluminum panel such that an adhesive layer surface protrudes, while pulling in the long side direction at a force of 1000 gf.

Next, another portion was fixed by a tape such that a protruding length of the adhesive layer of the samples set by the aforementioned methods (i) to (iii) is 25 mm in the long side direction. Finally, a piece of a commercially available disposal bag for a diaper (A deodorizing bag for diapers manufactured by Kurilon Chemicals Japan) cut to a size of 50 mm×100 mm was adhered onto the samples using a 2 kg roller, and then the 90 degree peeling force was measured under a 300 mm/min rate condition using a tensile tester. The measurement results of the samples are shown in the following Table 4. As shown in the following Table 4, the adhesive force (90 degree peeling force) after stretching Example 1 was confirmed to be lower than the adhesive force of Example 1 before stretching and the adhesive force of Comparative Example 1, and that the occurrences of adhesion to another article or the like other than a target article was suppressed.

TABLE 4

| | Example 1 | Example 1 | Comparative |
|---|---|---|---|
| | (Before stretching) | (After stretching) | Example 1 |
| Adhesive force | 1.5 | 0.6 | 1.6 |
| (N/25 mm) | | | |

### REFERENCE NUMERALS

1, 1A, 1B, 1C First strip member
2, 2A, 2B, 2C Second strip member
2a First end portion
2b Second end portion
3 Third strip member
5 First bonding portion
6, 6A Second bonding portion
10, 10A, 10B, 10C, 10D, 10E, 10F Securing member
11, 11A Substrate layer (first substrate layer)
11a, 21a, 31a Surface (first surface)
11b, 21b, 31b Surface (second surface)
12 Adhesive layer (first adhesive layer)
13 Tab portion
14 Latching portion
15 Outermost layer
21, 21A Substrate layer (second substrate layer)
22 Adhesive layer (second adhesive layer)
23 Penetrating portion
23A Opening portion (penetrating portion)
31 Substrate layer (third substrate layer)
32 Adhesive layer (third adhesive layer)
100 Disposable diaper
A Longitudinal direction
B Width direction

1. A securing member, comprising:
a first strip member;
a second strip member;
a third strip member;
a first bonding portion of the second strip member and first strip member; and
a second bonding portion of the second strip member and third strip member;
the first to third strip members being folded and laminated; wherein
the first strip member has a strip-shaped first substrate layer and a first adhesive layer provided on a first surface of the first substrate layer;
the second strip member has a strip-shaped second substrate layer positioned between the first strip member and the third strip member, and a second adhesive layer provided on a first surface of the second substrate layer;
the third strip member has a strip-shaped third substrate layer and a third adhesive layer provided on a first surface of the third substrate layer;
at least a portion of the first adhesive layer is detachably adhered to a second surface of the second substrate layer,
at least a portion of the second adhesive layer is detachably adhered to a second surface of the third substrate layer, and
the second substrate layer has elasticity, and a width of at least a portion thereof narrows when a tensile load is applied in one direction.

2. The securing member according to claim 1, wherein when the second substrate layer stretches 300% along the one direction, the degree of reduction of the width of the second substrate layer is 20% to 60%.

3. The securing member according to claim 1, wherein a load when the second substrate layer is stretched 200% along the one direction is from 0.5 N/25 mm to 20 N/25 mm.

4. The securing member according to claim 1, wherein a load when the second substrate layer is stretched 300% along the one direction and then returned to 200% is from 0.1 N/25 mm to 10 N/25 mm.

5. A securing member, comprising:
a first strip member;
a second strip member;
a third strip member;
a first bonding portion of the second strip member and first strip member; and
a second bonding portion of the second strip member and third strip member;
the first to third strip members being folded and laminated; wherein
the first strip member has a strip-shaped first substrate layer and a first adhesive layer provided on a first surface of the first substrate layer;
the second strip member has a strip-shaped second substrate layer positioned between the first strip member and the third strip member;
the third strip member has a strip-shaped third substrate layer and an adhesive layer provided on a first surface of the third substrate layer; and
at least a portion of the first adhesive layer is detachably adhered to the second substrate layer and a second surface of the third substrate layer.

6. The securing member according to claim 5, wherein the second substrate layer can be deformed by applying a tensile load.

7. The securing member according to claim 1, wherein a penetrating portion along a laminating direction of the first to third strip members is provided on the second substrate layer, and
the first adhesive layer is detachably adhered to the second surface of the third substrate layer through the penetrating portion.

8. The securing member according to claim 1, wherein the first strip member is provided on the first surface of the first substrate layer, and further provides a latching portion that can latch onto a member to be secured.

9. The securing member according to claim 8, wherein the securing member has a tab portion positioned on a second end portion on a side opposite from a first end portion configuring the first bonding portion on the first strip member, and
the latching portion is disposed to not overlay on the tab portion, first bonding portion, and second bonding portion, in a laminating direction in a condition where the first to third strip members are laminated.

10. The securing member according to claim 8, wherein
the securing member has a tab portion positioned on the second end portion on the side opposite from the first end portion configuring the first bonding portion on the first strip member, and
the latching portion is disposed between the tab portion and first bonding portion with 3 mm to 20 mm intervals from an end portion of a first bonding portion side of the tab portion.

11. The securing member according to claim 8, wherein the securing member is latched on the member to be secured by a 90 degree peeling force of 0.5 N/25 mm or greater, by the latching portion and first adhesive layer.

12. The securing member according to claim 8, wherein the securing member is latched on the member to be secured by a dynamic shear force of 4N/(25 mm×25 mm) or greater, by the latching portion and first adhesive layer.

13. The securing member according to claim 1, wherein a long side length in a condition where the first to third strip members are laminated is from 5 mm to 50 mm.

14. The securing member according to claim 1, wherein an uneven surface is provided on the second surface of the first substrate layer.

15. An absorbent article, comprising:
the securing member according to claim 1; and
a member to be secured where the securing member is adhered.

16. The absorbent article according to claim 15, wherein
the member to be secured is a diaper, and
after leaving to stand for 1 hour in a condition where 200 mL of 40° C. water was absorbed by the diaper, left to stand for 2 minutes, an opened securing member was wound on the diaper while pulling at a force of 500 gf, and the first adhesive layer is adhered to the diaper, the adhering conditions of the securing member to the diaper is maintained.
